(19) United States
(12) Patent Application Publication
(10) Pub. No.: US 2012/0165692 A1
(43) Pub. Date: Jun. 28, 2012

(54) IMPLANTABLE MEDICAL DEVICE AND A METHOD FOR USE IN AN IMPLANTABLE MEDICAL DEVICE

(76) Inventors: Malin Hollmark, Solna (SE); Andreas Karlsson, Solna (SE)

(21) Appl. No.: 13/334,735

(22) Filed: Dec. 22, 2011

Related U.S. Application Data
(60) Provisional application No. 61/437,725, filed on Jan. 31, 2011.

(30) Foreign Application Priority Data
Dec. 22, 2010 (EP) 10196637.2

(51) Int. Cl. A61B 5/046

(52) U.S. Cl. 600/518

(57) ABSTRACT

An implantable medical device is connectable to at least three electrodes, and includes an immittance measurer that performs immittance measurements within the heart of a patient using at least three electrodes coupled to the device, with at least one of the electrodes is arranged in an atrium of the patient's heart. The medical device further includes an immittance converter that converts the immittance measurement values into individual near-field immittance values of the at least one electrode arranged in an atrium, an atrial dilatation detector that detects atrial dilatation based upon the individual near-field immittance values, and that determines atrial dilatation values in dependence thereon, and an atrial fibrillation risk determiner that determines an atrial fibrillation risk index based upon the atrial dilatation values.

### RELATED APPLICATION

The present application claims the benefit of the filing date of Provisional Application 61/437,725, filed on Jan. 31, 2011.

### BACKGROUND OF THE INVENTION

1. Field of the Invention

The present invention relates to a device and a method according to the preambles of the independent claims, and in particular to a device and a method adapted to determine a risk of atrial fibrillation.

2. Description of the Prior Art

Atrial fibrillation is a very common arrhythmia. During episodes of atrial fibrillation, the systolic function of the atria is lost. This results in dilatation of the atria which in turn makes it more difficult for the heart to return to sinus rhythm. Without regular systolic activity the atria will only be passive mediators of blood volume to the ventricles. The degree of dilatation of the atria will reflect the venous return, i.e. preload.

Recent experimental animal studies have demonstrated that the right atrial (RA) stretch and dilatation can lead to development of atrial fibrillation (AF) (Ravelli 2003, Mechano-Electric Feedback and Cardiac Arrhythmias, Progress in Biophysics and Molecular Biology, 82(1-3)137-149). In addition, RA dilatation follows left atrial (LA) dilatation and vice versa. Thus, monitoring of the volume of one atrium will provide monitoring of the other.

WO-2004/028629 relates to a heart stimulator detecting atrial arrhythmia by determining wall distension by impedance measurement. Upon detection of an atrial arrhythmia the stimulation mode is switched and the pacing rate is adapted to limit the atrial distension. The heart stimulator may also be used for monitoring the degree of atrial distension over an extended period of time to be able to follow the disease development and to enable the physician to adapt therapy accordingly.

In a research paper (“Effects of spironolactone on atrial structural remodeling in a canine model of atrial fibrillation produced by prolonged atrial pacing”, J Zhao et al, British Journal of Pharmacology (2010), 159, pp 1584-1594) it is briefly discussed the generally accepted fact that atrial fibrillation (AF) and atrial dilatation may be mutually dependent and constitute a vicious circle. LA dilatation has been identified as an independent risk factor for the development of AF. AF results in progressive atrial dilatation, which in turn, might contribute to the self-perpetuating nature of arrhythmia. Atrial dilatation is due to an increase in atrial compliance caused by atrial contractile dysfunction during AF. An increase in atrial size will facilitate the development of atrial fibrillation. Furthermore, an elevated intra-atrial pressure will increase atrial wall stress, which may result in heterogeneities in conduction. In addition, atrial dilatation may promote focal arrhythmias that trigger self-perpetuating AF or maintain irregular atrial activation by mechano-electric feedback. The increased inhomogenity in atrial electrophysiological properties during atrial dilatation contributes to the development of AF. According to the presented data, interventions targeting a reduction of LA size may prevent AF or AF disease progression.

### SUMMARY OF THE INVENTION

The inventors have identified a relationship between an increased atrial dilatation and the risk of developing atrial fibrillation, and an object of the present invention is to provide an improved device and method of determining atrial dilatation and, thus, the risk of developing atrial fibrillation.

The inventors have found that by using so-called near-field immittance measurements, local measurement values may be determined being specifically suitable for determining a measure of atrial dilatation.

Using the proposed approach the present invention aims at monitoring the heart chamber volumes using impedance. This enables dilatation monitoring and, ultimately, AF or AF disease progression prevention. Early dilatation detection prior to AF can deter the disease progression by early medical intervention.

In a dilated heart, the blood volume in the proximity of an electrode is larger and varies less during the heart cycle than in a normal healthy heart. Such differences between a dilated chamber and a healthy chamber can be detected by measuring and analysing the impedance signal from the chamber in question. Chamber dilatation is detected as a decreased average impedance as well as lower peak-to-peak variation of the impedance.

Thus, a recorded impedance waveform reflects the superposition of fluid volume around the electrode pair throughout the cardiac and respiratory cycles, As an example, when measuring e.g. the impedance between an RAring electrode (right atrial ring electrode) and a SVC (superior vena cava) electrode, it is possible to generate an algorithm for the calculation of fluid volume surrounding the RAring (the so-called RAring near-field), which in turn reflects the RA volume. Thus, impedance measurements associated with the RAring electrode reflects the RA volume. Since RA dilatation follows LA dilatation, monitoring of the RA volume will also provide a monitor of the LA volume.

Left-sided heart diseases often lead to increased left atrial (LA) pressure, which inevitably will lead to dilatation of LA and subsequently atrial fibrillation (AF). Early dilatation detection prior to AF will help pacemaker and CRT (cardiac resynchronization therapy) patients by deterring the disease progression through early medical intervention. The present invention suggests ambulatory monitoring of the right atrial (RA) volume by impedance measurements. Since RA dilatation follows LA dilatation, monitoring of the RA volume will also provide a monitor of the LA volume. Impedance measurement of the fluid displacement in the immediate volume surrounding the RAring will provide a measure of dilatation.

In addition to provide an AF risk indication the present invention also may provide measures to monitor the progression of AF.

### LIST OF ABBREVIATIONS USED HEREIN

RA right atrium
LA left atrium
RV right ventricle
LV left ventricle
RAring/RAring electrode right atrial ring electrode
RAtip/RAtip electrode right atrial tip electrode
LAring/LAring electrode left atrial ring electrode
LAtip/LAtip electrode left atrial tip electrode
RVring/RVring electrode right ventricular ring electrode
RVtip/RVtip electrode right ventricular tip electrode
LVring/LVring electrode left ventricular ring electrode
LVtip/LVtip electrode left ventricular tip electrode
RV coil right ventricular coil electrode
SVCoil superior vena cava coil electrode

### BRIEF DESCRIPTION OF THE DRAWINGS

FIG. 1 is a schematic block diagram illustrating an implantable medical device according to the present invention.

FIG. 2 illustrates the principle for calculation of near-field immittance values.

FIG. 3 is a flow-diagram illustrating a method according to the present invention.

FIGS. 4 and 5 illustrate two different electrode set-ups which both would be applicable in relation to the present invention.

FIG. 6 shows a graph illustrating schematic impedance signals.

FIG. 7 shows a graph illustrating impedance signals from a pre-clinical study.

### DESCRIPTION OF THE PREFERRED EMBODIMENTS

State-of-the art implantable medical devices are often equipped to measure impedance (or related electrical parameters such as admittance) between various pairs of electrodes implanted within the patient. Examples include intracardiac impedance measurements made between pairs of electrodes mounted to leads implanted on or within the various chambers of the heart. Other examples include intrathoracic impedance measurements made between the housing of the device (or “can” or “case”) and electrodes implanted on or within the heart. Traditionally, such impedance measurements were deemed to be representative of the electrical impedance between the electrodes. That is, impedance measurements were associated with a particular pair of electrodes or some combination of three or more electrodes. Herein, these measurements are generally referred to as normal impedance measurements, or only “impedance measurements”, because the measurements are associated with at least one pair of electrodes. In terms of analyzing and interpreting the measured impedance data, the interpretation typically relied on a conceptual model wherein the measured impedance was deemed to be representative of the impedance of the field between the electrode pairs, including far-field contributions to that impedance. Under the far-field model, impedance measured between a pair of electrodes A and B is deemed to be representative of the field between A and B.

As one example of the far-field model, intrathoracic impedance measurements made between the device housing and a cardiac electrode implanted within the heart are deemed to represent the impedance to electrical flow spanning a field extending through the lungs between the device and the cardiac electrode. This intrathoracic impedance measurement may then be used to, for example, assess pulmonary fluid congestion to detect pulmonary oedema (PE) or heart failure (HF). Although this traditional interpretation of the impedance measurements can be useful, it has been recognized that an alternative interpretation of impedance measurements based on a “near-field model” can provide a more useful means for understanding, analyzing and interpreting impedance measurements.

The present invention is generally directed to the near-field impedance model and various systems, methods and applications that exploit this model.

In accordance with exemplary embodiments of the invention, an implantable medical device, such as a pacemaker, an implantable cardioverter defibrillator (ICD) or a cardiac resynchronization therapy (CRT) device, and a method for use in an implantable medical device, are provided for determining and exploiting near-field immittance values (wherein “immittance” broadly refers to impedance, conductance, admittance or other generally equivalent electrical values or parameters) associated with individual electrodes in accordance with a near-field model that associates immittance values with individual electrodes rather than with pairs of electrodes.

In this regard, exemplary techniques provided herein exploit the aforementioned near-field model, which offers a new perspective for the interpretation of the immittance measurements that significantly simplifies the analysis and interpretation of data and the development of detection methods/procedures. Briefly, the near-field model is based on the recognition that the immittance between a pair of electrodes (A and B) can be modelled as a superposition of near-field immittance values that are associated with the individual electrodes (i.e. A+B). That is:

Traditional model: Immittance=A to B=Field between A and B

Near-field model: Immittance=A+B=Near-field A+Near-field B More generally, the near-field model transforms multiple pair-based immittance measurement values into a set of near-field immittance values that can be interpreted and analyzed more easily. In an example where impedance is measured, the conversion of normal impedance measurement values into near-field impedance values is performed by converting N (where N is at least three) impedance measurement values (v1 v2, . . . , vN) into a set of linear equations to be solved whereby far-field contributions to impedance are ignored. The set of linear equations are then solved to yield a set of near-field impedance values (e1, e2, . . . , eN) associated with the individual electrodes. In other examples, N+1 impedance measurements (or some even larger number) are used to determine the near-field impedance values of the N electrodes.

One important advantage of the near-field model is that by deriving near-field immittance values associated with individual electrodes, the device can easily associate a specific physical entity—such as the particular anatomical structure adjacent to the electrode—with the corresponding near-field immittance value.

For example, for an RAring electrode, the corresponding near-field immittance is associated with the local fluid and tissue content within the adjacent RA cavity and RA tissues.

The basis of the algorithm used in the present invention is that when several immittance configurations are measured, each current node reflects the tissue-to-blood proportionality in its immediate surrounding. As an example of this, the following configuration (with reference to FIG. 2) may be used:

v1: RAring (shown as A in FIG. 2)—case (C)

v2: RAring (A)—LVring (B)

v3: LVring (B)—case (C)

These equations form an “impedance triangle” which may be solved for each node by the following equation systems:

System 1:

v1=A+C

v2=A+B

v3=B+C

System 2:

v1+v2-v3=2A=2 RAring

v2+v3-v1=2 B=2 LVring

v1+v3-v2=2C=2 case

Thus, the three impedance waveforms measured with the three impedance fields are in fact composites made up of the three distinct waveforms from each of the three nodes, A, B and C. The three distinct waveforms are extracted by using the equation system 2 above.

Consequently, measurement of the impedances suggested above (or any other impedances that include the RAring and/or RAtip), but still creating an “impedance triangle”, will provide an estimation of the fluid volume surrounding the RAring or RAtip. This, in turn, reflects the RA volume.

When performing immittance measurements bipolar configurations are not a prerequisite, quadrupolar configurations may be used as well.

For instance, the quadrupolar configuration: I: RAring-LVring, U: RAtip-LVtip could replace the bipolar configuration I: RAring-LVring, U: RAring-LVring (where I denotes the current injection nodes and U the voltage nodes).

Additionally, and within the scope of the present invention as defined by the claims, it is possible to measure impedance (immittance) over more than three anatomical locations. For example, the measurements may be performed by using geometries with four poles (e.g. RAring/RAtip, LAring(s)/LAtip, RVring/RVtip and Case). This would provide mean impedance values for the electrodes in the measured configurations.

If one specific electrode would be of special interest, this electrode may be measured against two larger electrodes. The surface ratio together with the near-field model evaluation would then ensure that the electrode of specific interest would have a significant signal contribution. The triangle could then e.g. include the following configurations: RAring-Case, RAring-RVcoil, RVcoil-Case; where the RAring-electrode is of particular interest.

The present invention will now be described in more detail with references to the block diagram shown in FIG. 11.

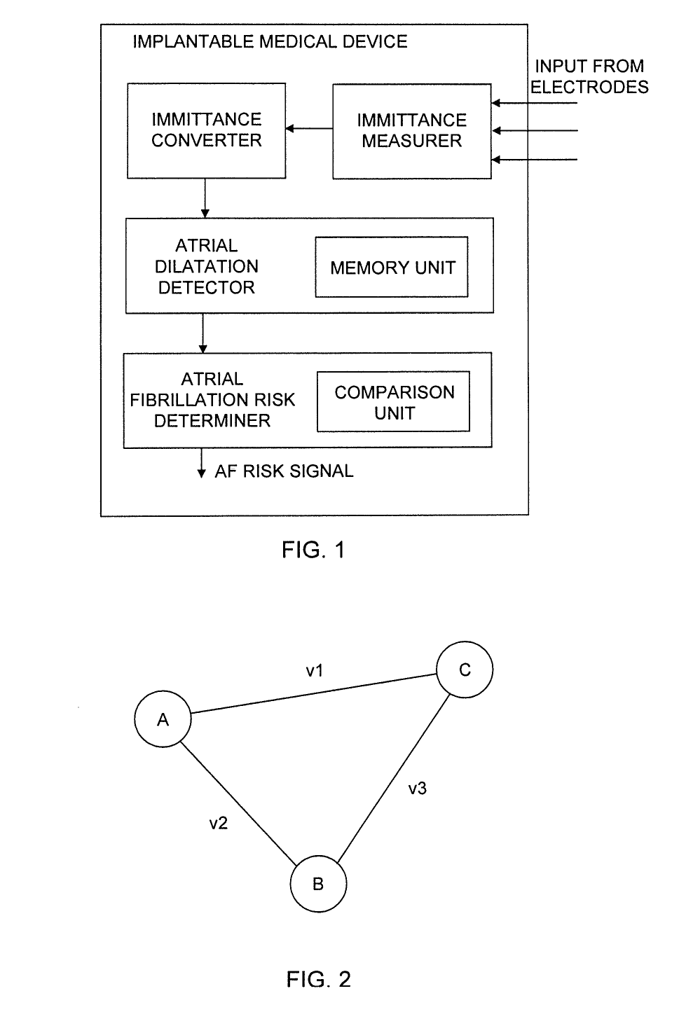

In FIG. 1 is schematically shown an implantable medical device according to the invention. The implantable medical device is connectable to at least three electrodes. The electrodes, to which the implantable medical device is connectable, may, for example, be selected from the group of: the case (or can) of the implantable medical device, RAring electrodes, an RAtip electrode, LAring electrodes, an LAtip electrode, LVring electrodes, an LVtip electrode, RVring electrodes, an RVtip electrode, RVcoil electrodes or SVCoil electrodes. In FIG. 1 the input signals from the electrodes are indicated by three parallel arrows. The implantable medical device comprises an immittance measurer operative to perform immittance measurements within the heart of a patient using at least three of said electrodes where at least one of the electrodes is arranged in an atrium of the patient's heart.

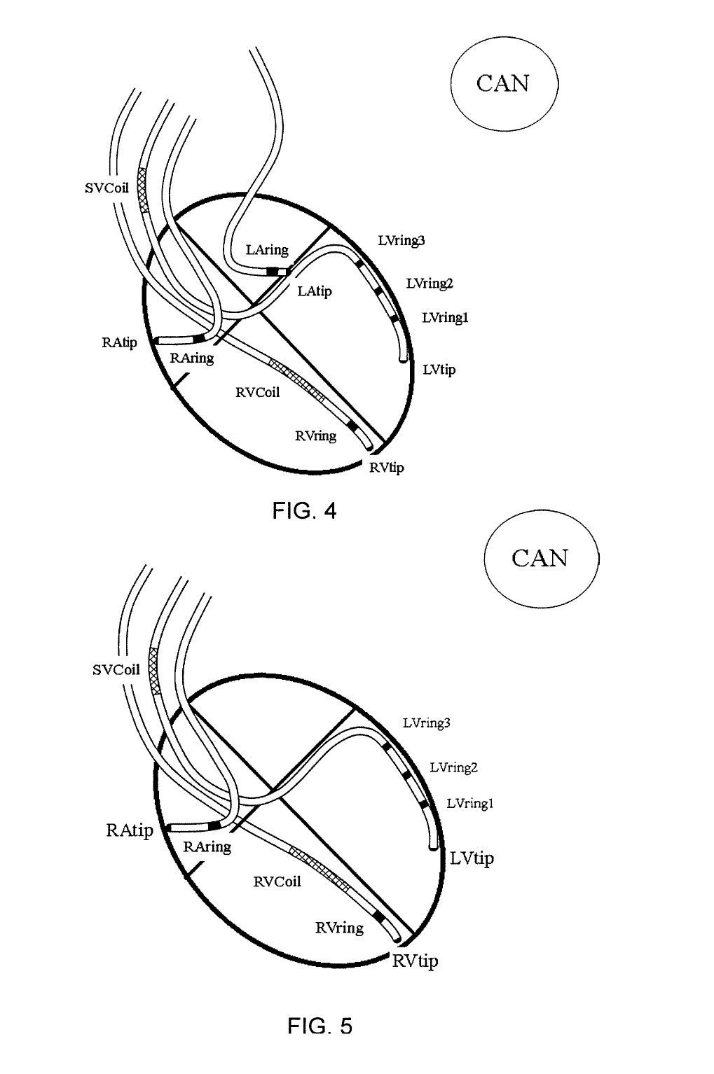

FIGS. 4 and 5 illustrate two different electrode set-ups which both would be applicable in relation to the present invention.

The medical device further comprises an immittance converter operative to convert immittance measurement values into individual near-field immittance values of at least one of the at least one electrode being arranged in an atrium, The device in addition comprises an atrial dilatation detector operative to detect atrial dilatation based upon the individual near-field immittance values, and to determine atrial dilatation values in dependence thereto. An atrial fibrillation risk determiner is also included in the device, which risk determiner is adapted to determine an atrial fibrillation risk index based upon the atrial dilatation values.

Preferably, the atrial fibrillation risk determiner is adapted to generate an atrial fibrillation risk signal in dependence of the risk index.

The atrial dilatation detector may also comprise a memory unit for storage of the determined atrial dilatation values.

Furthermore, the atrial fibrillation risk determiner may comprise a comparison unit provided with at least one atrial fibrillation risk threshold. The comparison unit is adapted to compare the determined atrial dilatation values with the at least one fibrillation risk threshold and the atrial fibrillation risk determiner is adapted to determine the atrial fibrillation risk index in dependence of the comparison. The atrial fibrillation risk threshold is an atrial dilatation value for which the risk of atrial fibrillation is considered to be significant.

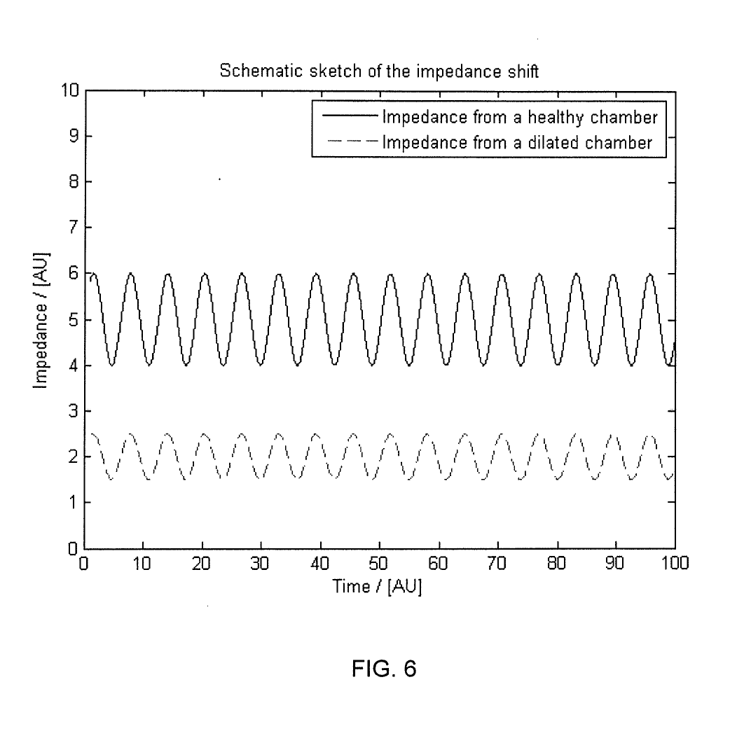

Returning to the graphs shown in FIG. 6 where the upper curve shows the impedance from a healthy heart chamber and the lower curve shows the impedance from a dilated heart chamber, e.g. from a right atrial ring electrode. Two significant differences between the curves may be observed. One difference is the DC-level, which is higher for the healthy heart and which is related to the smaller volume of the heart chamber. The DC-level is the average of the measured impedance. Another difference is the AC-amplitude, which is smaller for the dilated chamber than for the healthy heart chamber. This is caused by the inelasticity of the heart wall during AF. The AC-amplitude is the peak-to-peak variation of the impedance. These two parameters, the DC-level and the AC-amplitude, are advantageously used as parameters for the atrial fibrillation risk thresholds.

Preferably, the determined atrial fibrillation risk index is based upon the variations of the determined atrial dilatation values during a preset time period. The atrial dilatation variation during healthy periods can also be considered when setting the thresholds for what is to be considered a pathological change of the atrial dilatation. Thus, the risk index may be based upon the variations of the determined atrial dilatiation values.

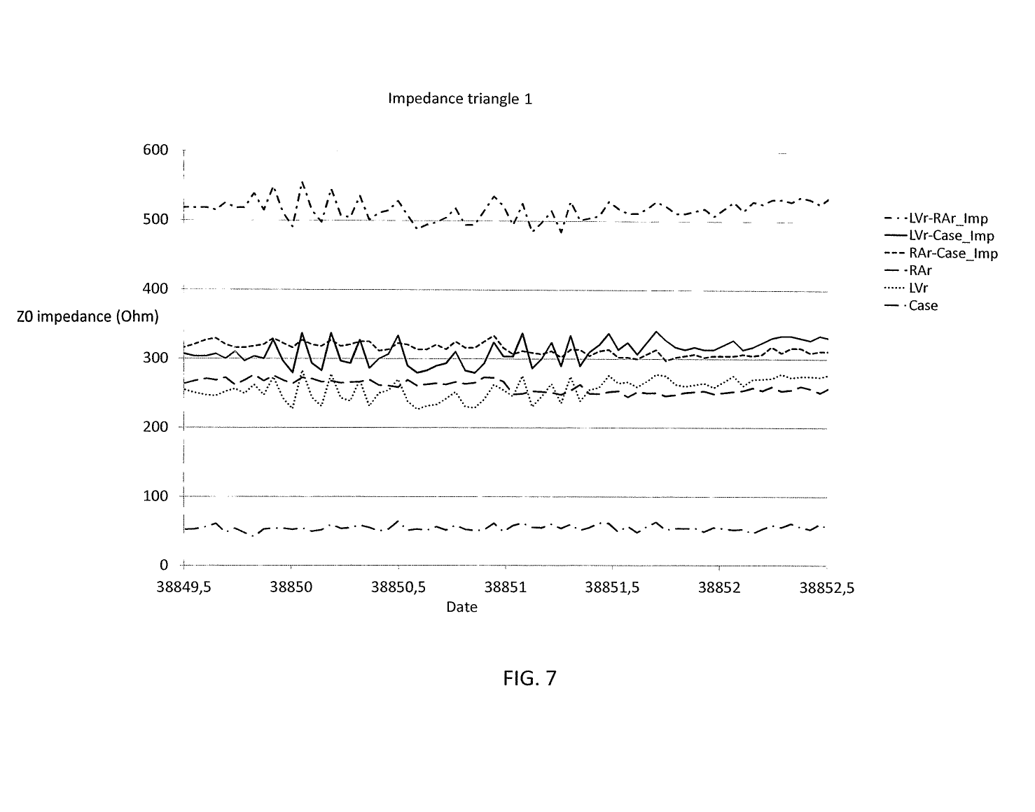

A measurement session, during which the immittance measurements are performed, has preferably a duration of some seconds, at least one respiration cycle or a number of heart cycles, and is performed at regular intervals, e.g. once every hour or every two hours. By storing the determined atrial dilatation values in the memory unit it will be possible to identify both short-term and long-term changes. The graphs illustrated in FIGS. 7 and 8 show impedance values during three days and may be regarded to show short-term changes. Long-term changes may be identified during time periods of weeks, months or even years.

In one embodiment one atrial electrode is an RAring electrode. FIGS. 4 and 5 show an RAring electrode arranged in the right atrium. The immittance measurement of the fluid volume surrounding the RAring will provide a measure of dilatation of the right atrium.

In another embodiment one atrial electrode is an LAring electrode and the immittance measurement of the fluid volume surrounding the LAring will provide a measure of dilatation of the left atrium. This is also illustrated in FIG. 4.

As discussed above at least three electrodes are required to perform the immittance measurements.

One specific embodiment of the present invention is achieved when the immittance measurements are made between electrodes that correspond to an impedance triangle, i.e. when the immittance measurements are performed with measurement nodes arranged in a triangle.

In all cases, conversion of the immittance measurement values into relative near-field immittance values is achieved, by the immittance converter, by ignoring far-field contributions.

This is achieved, as discussed above, by converting the immittance measurement values into near-field immittance values, by the immittance converter, by converting at least N immittance measurement values (v1, v2, . . . , vN) into a set of linear equations to be solved while ignoring the far-field contributions to the immittance measurements, where N is at least three; and by solving the set of linear equations to yield a set of near-field immittance values (e1, e2, . . . , eN).

Thus, the immittance converter is adapted to convert the immittance measurement values into relative near-field immittance values by ignoring far-field contributions. More specifically, the immittance converter is adapted to convert the immittance measurement values into near-field immittance values by converting at least N immittance measurement values (v1, v2, . . . , vN) into a set of linear equations to be solved while ignoring the far-field contributions to the immittance measurements, where N is at least three, and by solving the set of linear equations to yield a set of near-field immittance values (e1, e2, . . . , eN).

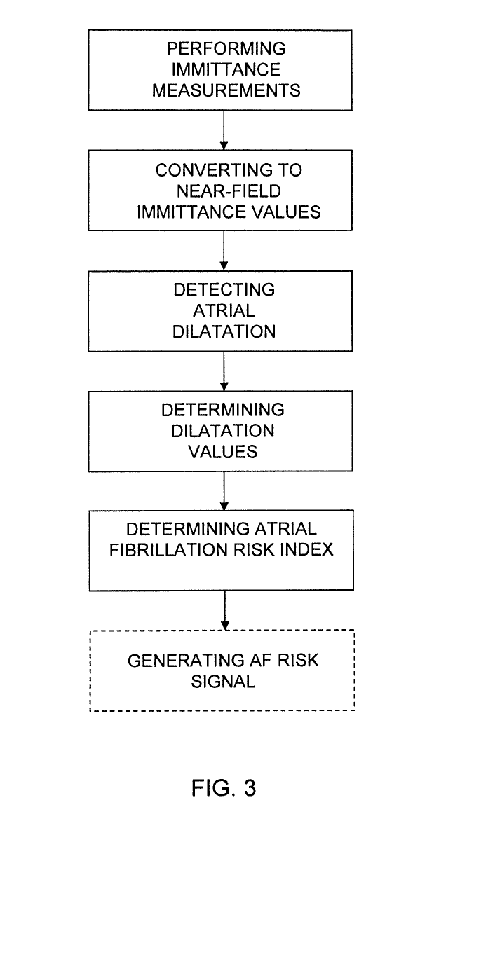

With reference to FIG. 3, the present invention also relates to a method for use in an implantable medical device for implantation within a patient.

The method comprises:
performing immittance measurements within the heart of the patient using at least three electrodes connected to the device, where at least one electrode is arranged within an atrium of the patient;
converting the immittance measurement values to individual near-field immittance values for at least one of said at least one electrode arranged within an atrium;
detecting atrial dilatation based upon the near-field immittance values, and determining atrial dilatation values in dependence thereto, and
determining an atrial fibrillation risk index based upon said atrial dilatation values.

In the figure is also included, as an optional step, that the method includes generating an AF risk signal in dependence of said risk index.

The method may further include comparing the determined atrial dilatation values with at least one atrial fibrillation risk threshold and generating the atrial fibrillation risk index in dependence of the comparison. The determined atrial fibrillation risk index is based upon the variations of the determined atrial dilatation values during a preset time period, which is discussed in detail above.

Preferably, one of the at least one atrial electrode is an RAring electrode and the immittance measurement of the fluid volume surrounding the RAring will provide a measure of atrial dilatation.

In another embodiment one of the at least one atrial electrode is an LAring electrode and the immittance measurement of the fluid volume surrounding the LAring will provide a measure of atrial dilatation.

One specific embodiment of the present invention is achieved when the immittance measurements are made between electrodes that correspond to an impedance triangle, i.e. when the immittance measurements are performed with measurement nodes arranged in a triangle.

The conversion of the immittance measurement values into relative near-field immittance values is achieved by ignoring far-field contributions to the impedance measurements, which specifically is achieved by:

converting at least N immittance measurement values (v1, v2, . . . , vN) into a set of linear equations to be solved while ignoring the far-field contributions to the immittance measurements, where N is at least three, and solving the set of linear equations to yield a set of near-field immittance values (e1, e2, . . . , eN).

The method preferably includes the step of controlling at least one device function in response to the near-field immittance values. This may be a specifically tailored stimulation mode adapted to reduce the effect of AF, or even to prevent the occurrence of AF.

FIGS. 4 and 5 illustrate two different electrode set-ups which both would be applicable in relation to the present invention. A lead located in the left atrium, as well as in the right, would provide near-field impedance from that specific location and the near-field impedance signals from both atria can be monitored. This would indicate where the dilatation and therefore the fibrotic tissue reside. The fibrosis of tissue is a consequence of the atrial remodelling, which, in turn, is caused by the activation of hormone systems as a response to the atrial dilatation. The fibrotic tissue might be the substrate for AF and subsequently the origin of AF. A dilatation originating in the RA may be indicative of a right-sided disease, such as pulmonic or tricuspid valve stenosis, pulmonary disease or chronic obstructive pulmonary disease (COPD). A dilatation originating in the LA may be indicative of e.g., aortic or mitralis valve stenosis or systemic hypertension. The invention could thus provide an improved monitoring of disease progression and dilatation/AF.

FIG. 6 shows a graph illustrating impedance signals from a healthy heart chamber (upper curve) that will have a higher DC level and larger peak-to-peak amplitude than a signal originating from a dilated chamber (lower curve).

FIG. 7 shows impedance signals from a pre-clinical study. The upper lines are the recorded signals from three Z configurations in an impedance triangle 1. By references to FIG. 4 the impedance triangle is formed between left ventricle ring electrode (LVr) which could be one of LVring1, LVring2 or LVring3 in FIG. 4, right atrial ring electrode (RAr) which is denoted RAring in FIG. 4, and the case. The three lower lines are the near-field signals for the respective electrode extracted through the equation system outlined above.

According to one embodiment the relationship between different anatomical regions in the heart may be reflected by the signals obtained from one or several impedance triangles. For instance, by comparing the different terms (electrode nodes) in one impedance triangle, or the relation between electrode nodes from several impedance triangles, e.g. the configuration RAring-LVring included in the impedance triangle referred to in relation to FIG. 7, and another impedance triangle formed e.g. by the electrodes RAring, LVring3 and the case (can) by references to FIG. 4 or 5. The two different RAring signals will be extracted from two equation systems formed by the two impedance triangles and these signals will reflect the near-field in the RA produced by two different configurations.

The present invention is not limited to the above-described preferred embodiments. Various alternatives, modifications and equivalents may be used. Therefore, the above embodiments should not be taken as limiting the scope of the invention, which is defined by the appending claims.

Although modifications and changes may be suggested by those skilled in the art, it is the intention of the inventors to embody within the patent warranted hereon all changes and modifications as reasonably and properly come within the scope of their contribution to the art.

We claim as our invention:

1. An implantable medical device connectable to at least three electrodes, said implantable medical device comprising:
an immittance measurer configured to perform immittance measurements within the heart of a patient using at least three of said electrodes, with at least one of said electrodes arranged in an atrium of the heart;
an immittance converter configured to convert immittance measurement values into individual near-field immittance values of said at least one of said electrodes arranged in an atrium;
an atrial dilatation detector configured to detect atrial dilatation based upon said individual near-field immittance values, and to determine atrial dilatation values in dependence thereto; and
an atrial fibrillation risk determiner configured to determine an atrial fibrillation risk index based upon said atrial dilatation values.

2. The implantable medical device according to claim 1, wherein said atrial fibrillation risk determiner is configured to generate an atrial fibrillation risk signal in dependence of said risk index.

3. The implantable medical device according to claim 1, wherein the atrial dilatation detector comprises a memory unit for storage of said determined atrial dilatation values.

4. The implantable medical device according to claim 1, wherein said atrial fibrillation risk determiner comprises a comparison unit provided with at least one atrial fibrillation risk threshold, said comparison unit is configured to compare said determined atrial dilatation values with said at least one atrial fibrillation risk threshold to obtain a comparison result, and said atrial fibrillation risk determiner is configured to determine said atrial fibrillation risk index in dependence on the comparison result.

5. The implantable medical device according to claim 1, wherein said atrial fibrillation risk determiner is configured to determine the atrial fibrillation risk index based on variations of the determined atrial dilatation values during a preset time period.

6. The implantable medical device according to claim 1, wherein said at least one of said electrodes is a right atrial ring electrode.

7. The implantable medical device according to claim 1, wherein said at least one of said electrodes is a left atrial ring electrode.

8. The implantable medical device according to claim 1, wherein said immittance measurer is configured to perform said immittance measurements with measurement nodes arranged in a triangle.

9. The implantable medical device according to claim 1, wherein said immittance converter is configured to convert the immittance measurement values into relative near-field immittance values by ignoring far-field contributions.

10. The implantable medical device according to claim 9, wherein said immittance converter is configured to convert the immittance measurement values into near-field immittance values by converting at least N immittance measurement values (v1, v2, . . . , vN) into a set of linear equations to be solved while ignoring the far-field contributions to the immittance measurements, where N is at least three and by solving the set of linear equations to yield a set of near-field immittance values (e1, e2, . . . , eN).

11. A method for use in an implantable medical device for implantation within a patient, the method comprising:
performing immittance measurements within the heart of the patient using at least three electrodes connected to the device, with at least one electrode arranged within an atrium of the patient;
converting immittance measurement values to individual near-field immittance values for said at least one of said electrodes arranged within an atrium;
detecting atrial dilatation based upon said near-field immittance values, and determining atrial dilatation values in dependence thereon; and
determining an atrial fibrillation risk index based upon said atrial dilatation values.

12. The method according to claim 11, comprising generating an atrial fibrillation risk signal in dependence of said risk index.

13. The method according to claim 11, comprising comparing the determined atrial dilatation values with at least one atrial fibrillation risk threshold and generating said atrial fibrillation risk index in dependence of the comparison.

14. The method according to claim 11, comprising determining said atrial fibrillation risk index based on the variations of the determined atrial dilatation values during a preset time period.

15. The method according to claim 11, comprising arranging said at least one of said electrodes as a right atrial ring electrode.

16. The method according to claim 11, comprising arranging said at least one of said electrodes as a left atrial ring electrode.

17. The method according to claim 11, comprising performing said immittance measurements with measurement nodes arranged in a triangle.

18. The method according to claim 11, comprising converting the immittance measurement values into relative near-field immittance values by ignoring far-field contributions.

19. The method according to claim 18, comprising converting the immittance measurement values into near-field immittance values by:
converting at least N vector-based immittance measurement values (v1, v2, . . . , vN) into a set of linear equations to be solved while ignoring the far-field contributions to the immittance measurements, where N is at least three, and solving the set of linear equations to yield a set of near-field immittance values (e1, e2, . . . , eN).

20. The method according to claim 11, comprising controlling at least one device function in response to the near-field immittance values.
